(19) United States
(12) Patent Application Publication
(10) Pub. No.: US 2025/0152183 A1
(43) Pub. Date: May 15, 2025

(54) TORQUE TRANSMISSION TUBE AND SURGICAL INSTRUMENT

(71) Applicant: Micro-Tech (Nanjing) Co., Ltd., Nanjing (CN)

(72) Inventors: Zhi Tang, Nanjing (CN); Mingqiao Fan, Nanjing (CN); Chao Li, Nanjing (CN)

(21) Appl. No.: 19/022,266

(22) Filed: Jan. 15, 2025

(30) Foreign Application Priority Data
Jan. 17, 2024 (CN) 202410075234.6

(51) Int. Cl. A61B 17/16; A61B 17/00

(52) U.S. Cl. CPC A61B 17/1633; A61B 2017/0046

(57) ABSTRACT

A torque transmission tube and a surgical instrument, belongs to the technical field of medical instruments. The torque transmission tube includes at least one spiral wire harness layer formed by spirally winding a plurality of strands of wires, so that the torsional strength of a first tube body is ensured by utilizing the spiral structure of the plurality of strands of wires of the first tube body, causing a torque input from a first end to be transmitted to a second end more effectively; by providing a second tube body and connecting a third end of the second tube body with the second end, the torque of the second end may be effectively transmitted to a fourth end of the second tube body through the third end.

### CROSS-REFERENCE TO RELATED APPLICATION

This application claims priority to Chinese Patent Application No. 202410075234.6, filed on Jan. 17, 2024, which is hereby incorporated by reference in its entirety.

### TECHNICAL FIELD

The present application belongs to the technical field of medical instruments, and particularly relates to a torque transmission tube and a surgical instrument.

### BACKGROUND

With the continuous development of endoscopic technology, minimally invasive surgery is being accepted and favored by more and more doctors and patients because of its advantages of less trauma, quick recovery and less pain. In general minimally invasive surgery, an endoscope is used as a carrier to reach a position of lesion through a light source carried by the endoscope itself at a flexible angle, and then a corresponding treatment instrument is inserted through a cavity of the endoscope to realize the treatment of a wound.

For the surgical instruments used under traditional endoscopes, the torque is transmitted by rotating a handle and then driving by the handle an inner core to rotate.

However, the inner core is very thin, and the torque cannot be transmitted well, which is not convenient for doctors to operate.

### SUMMARY

Objectives of the application: embodiments of the present application provide a torque transmission tube, which aims to solve the technical problem that in the prior art, use of the inner core to transmit the torque is not effective and it is inconvenient for doctors to operate; another objective of the embodiments of the present application is to provide a surgical instrument including the torque transmission tube above.

Technical solution: embodiments of the present application provide a torque transmission tube, including:
a first tube body, having a first channel and corresponding first and second ends in an extending direction of the first channel, where the first tube body includes a plurality of strands of wires, each strand of wire spirally winds from the first end to the second end, the plurality of strands of wires spirally wind to form at least one spiral wire harness layer, the at least one spiral wire harness layer surrounds to form the first channel;
a second tube body, having a second channel and corresponding third and fourth ends in an extension direction of the second channel, where the third end is connected to the second end, and the second channel is communicated with the first channel; and
where a minimum bending radius of the second tube body is smaller than a minimum bending radius of the first tube body.

In some embodiments, the first tube body includes a spiral wire harness layer, the plurality of strands of wires are arranged around the first channel and extend in a same direction to form the spiral wire harness layer.

In some embodiments, the first tube body includes a plurality of spiral wire harness layers, the plurality of spiral wire harness layers are stacked along a radial direction of the first tube body, and each of the spiral wire harness layers includes at least one strand of the wire.

In some embodiments, the wires of at least two adjacent spiral wire harness layers have different spiral extension directions.

In some embodiments, torque transmission tube further includes:
a connecting piece, provided between the first tube body and the second tube body, the second end being connected with the third end through the connecting piece.

In some embodiments, the wire includes a first connector located at the first end and a second connector located at the second end, the first connectors of the plurality of strands of wires are connected to each other, and the second connectors of the plurality of strands of wires are connected to each other.

In some embodiments, the extending direction of the first channel is an axial direction of the first tube body;
under an axial tensile force of 10 N, an axial tensile displacement of the first tube body does not exceed 5 mm; and
under an axial pressure of 60 N, an axial compression displacement of the first tube body does not exceed 40 mm.

In some embodiments, the number of the wires is n strands, a diameter of the wires is d mm, meeting: n/d≥1.

In some embodiments, the torque transmission tube further meets:

$$2 \leq n \leq 16 ; and 0 < d \leq 2 .$$

In some embodiments, the minimum bending radius of the first tube body is r₁, and the minimum bending radius of the second tube body is r₂, meeting: 1<r₁/r₂≤8.

In some embodiments, the torque transmission tube further meets:

$$20 mm \leq {r}_{1} \leq 40 mm ; and 5 mm \leq {r}_{2} \leq 20 mm .$$

In some embodiments, a length of the first tube body is L₁, and a length of the second tube body is L₂, meeting: 0<L₂/L₁≤1.

In some embodiments, a length of the torque transmission tube is L₃, meeting 1550 mm≤L₃≤3000 mm; and/or,
a maximum outer diameter of the torque transmission tube is R and a minimum inner diameter is r, meeting: R≤4.2 mm, and r≥0.2 mm.

In some embodiments, a lead S of a spiral wire formed by a spiral extension of the wire is 3-30 mm.

In some embodiments, the wire has a cross section perpendicular to the spiral extension direction of the wire;
the cross section is circular, or,
the cross section is polygonal, and the polygon has four or more sides.

In some embodiments, materials of the first tube body and the second tube body are both metal.

Correspondingly, a surgical instrument according to embodiments of the present application is applied to an endoscope, and the surgical instrument includes:
the torque transmission tube as described above;
an operating handle, provided at the first end; and
an executing piece, provided at the fourth end;
where the operating handle is used for driving the torque transmission tube to rotate so as to transmit a torque to the executing piece.

In some embodiments, the executing piece includes:
a clamping seat, provided at the fourth end;
a clamping part, connected with the clamping seat; and
a pulling part, movably provided in the first channel and the second channel, and connected between the operating handle and the clamping part;
where, the operating handle is also used for driving the pulling part to move along an extending direction of the pulling part, to drive the clamping part to move, and to separate the clamping seat and the clamping part from the torque transmission tube and the pulling part.

Beneficial effect: compared with the prior art, the torque transmission tube of embodiments of the present application includes: a first tube body, having a first channel and corresponding first and second ends in an extending direction of the first channel, where the first tube body includes a plurality of strands of wires, each strand of wire spirally winds from the first end to the second end, the plurality of strands of wires spirally wind to form at least one spiral wire harness layer, the at least one spiral wire harness layer surrounds to form the first channel; a second tube body, having a second channel and corresponding third and fourth ends in an extension direction of the second channel, where the third end is connected to the second end, and the second channel is communicated with the first channel, where a minimum bending radius of the second tube body is smaller than a minimum bending radius of the first tube body. The first tube body of the torque transmission tube including at least one spiral wire harness layer formed by spirally winding a plurality of strands of wires causes that the torsional strength of the first tube body is ensured by the spiral structure of the plurality of strands of wires of the first tube body, so that the torque input from the first end may be transmitted to the second end more effectively; and providing the second tube body and connecting the third end of the second tube body with the second end cause that the torque of the second end may be effectively transmitted to the third end and transmitted to the fourth end of the second tube body through the third end. Therefore, in a first aspect, the torque transmission tube may effectively transmit the torque; furthermore, the minimum bending radius of the second tube body is smaller than the minimum bending radius of the first tube body, that is, the second tube body is easier to bend, and thus the angle may be easily adjusted in a narrow organ cavity. Therefore, in a second aspect, the torque transmission tube has better operation flexibility, which is beneficial to improving the operation accuracy of endoscope instruments and reducing the operation difficulty.

Compared with the prior art, the surgical instrument of embodiments of the present application is applied to an endoscope, and the surgical instrument includes: the preceding torque transmission tube, an operating handle connected to the first end, and an executing piece connected to the fourth end, where the operating handle is used for driving the torque transmission tube to rotate so as to transmit a torque to the executing piece. The surgical instrument may have all technical features and beneficial effects of the preceding torque transmission tube, especially the torque of the operating handle may be transmitted to the executing piece more accurately through the torque transmission tube, so that the precise control of the executing piece may be realized, the operation difficulty of doctors may be reduced, the operation efficiency may be improved, and the operation risk may be reduced.

### BRIEF DESCRIPTION OF DRAWINGS

To describe the technical solutions in embodiments of the present application more clearly, the following briefly introduces the accompanying drawings needed for describing the embodiments. Apparently, the accompanying drawings in the following description illustrate merely some embodiments of the present application, and persons of ordinary skill in the art may still derive other drawings from these accompanying drawings without creative effort.

FIG. 1 is a structural schematic diagram of a torque transmission tube according to an embodiment of the present application.

FIG. 2 is a dimensional schematic diagram of a torque transmission tube according to an embodiment of the present application.

FIG. 3 is a cross-sectional structural schematic diagram of a torque transmission tube according to a first embodiment of the present application, taken along line A-A in FIG. 1.

FIG. 4 is a cross-sectional structural schematic diagram of a torque transmission tube according to a second embodiment of the present application, taken along line A-A in FIG. 1.

FIG. 5 is a structural schematic diagram of a torque transmission tube according to a third embodiment of the present application.

FIG. 6 is a structural schematic diagram of a first tube body of the torque transmission tube according to the third embodiment of the present application.

FIG. 7 is a structural schematic diagram of the first tube body and a connecting piece of the torque transmission tube according to the third embodiment of the present application.

FIG. 8 is a cross-sectional structural schematic diagram of the connecting piece according to the third embodiment of the present application.

FIG. 9 is a structural schematic diagram of a surgical instrument according to an embodiment of the present application.

FIG. 10 is a partially enlarged structural schematic diagram of area A of FIG. 9.

FIG. 11 is a schematic diagram illustrating detection of displacement of a torque transmission tube under an axial pressure, according to an embodiment of the present application.

Reference numerals: 10—torque transmission tube; 100—first tube body; 101—first end; 102—second end; 110—first channel; 120—spiral wire harness layer; 121—wire; 1211—first connector; 1212—second connector; 122—cross section; 200—second tube body; 201—third end; 202—fourth end; 210—second channel; 300—connecting piece; 310—body; 311—first assembly part; 312—second assembly part; 320—third channel; 20—operating handle; 30—executing piece; 31—clamping seat; 32—clamping part; 33—pulling part.

### DESCRIPTION OF EMBODIMENTS

The following clearly and comprehensively describes the technical solutions in embodiments of the present application with reference to the accompanying drawings of the embodiments of the present application. Apparently, the described embodiments are merely some rather than all of embodiments of the present application. All other embodiments obtained by persons of ordinary skill in the art based on the embodiments of the present application without creative effort shall fall within the protection scope of the present application.

In the description of the present application, it should be understood that the terms “proximal end” and “distal end” are used with reference to a surgical operator (operator), where the “proximal end” is an end of the surgical tool closer to the operator and the “distal end” is an end thereof farther from the operator than “proximal end”, ie., the end closer to a patient. The directional or positional relationships indicated by terms “inside”, “outside”, “up”, “down” and so on are based on the directional or positional relationships shown in the accompanying drawings, and are only for convenience of describing the present application and simplifying the description, and do not indicate or imply that the apparatus or component referred to must have a specific direction, or be constructed and operated in a specific direction, and therefore they cannot be understood as a limitation of the present application. In the description of the present application, “multiple” means two or more, “at least one” means one, two or more, unless otherwise specifically defined, and “connect” can be direct connection, that is, one object is directly connected with another object, or indirect connection, that is, one object is connected with another object through other object.

With the development of modern medicine, endoscope, as a highly flexible surgical instrument, has been widely used in various types of surgeries. General endoscopes are divided into gastroscope, enteroscope, etc. according to the position of use, and according to the distance to the position of lesion, endoscopes are made into various lengths so as to adapt to the corresponding position of lesion. In actual operation, the endoscope is first inserted into mouth or anus of the human body to enter the human body, then through the light source carried by the endoscope itself, and under visual observation, the position and angle of the front end of the endoscope are adjusted to enable the endoscope to be continuously transported to the distal end, and then the endoscope reaches the wound site. For example, for general hemostasis wounds, products such as hemostatic clips and hemostatic forceps need to be inserted and reach the position of the wound site through the forceps channel of the endoscope, and then the instrument is rotated to adjust the rotation angle of the instrument to a suitable position, so as to realize hemostasis of the wound tissue. The applicant noted that the traditional hemostatic closure instruments used under endoscope all achieve the closure and hemostasis of the wound tissue by rotating the handle, driving by the handle the inner core, and then driving by the inner core the clamping part at front end. Because the size of the inner core of the instrument is very small, generally no more than 0.6 mm in diameter, the torque transmission effect is relatively poor, and especially when the length of the instrument itself is getting longer and longer, the torque transmission loss will be more serious. Additionally, some surgical instruments used under endoscope have multiple inner cores, making it impossible to transmit the torque through the rotation of the inner cores.

In view of this, embodiments of the present application provide a torque transmission tube 10 and a surgical instrument to overcome at least one of the above technical problems.

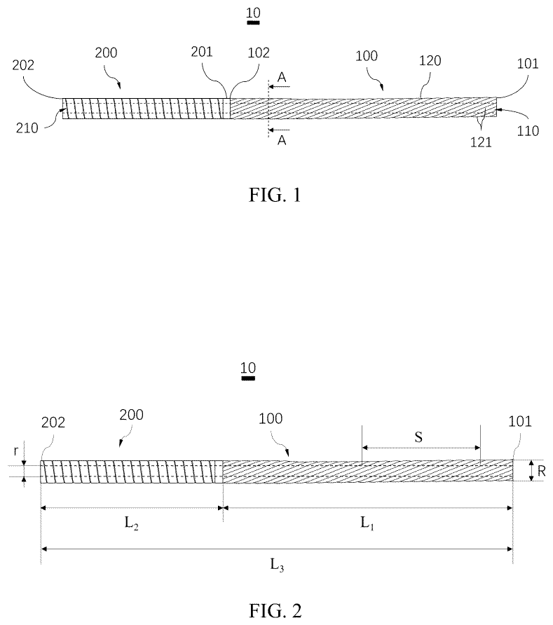

With reference to FIG. 1, the torque transmission tube 10 of an embodiment of the present application mainly includes a first tube body 100 and a second tube body 200. The first tube body 100 and the second tube body 200 are interconnected, and the first channel 110 of the first tube body 100 and the second channel 210 of the second tube body 200 are intercommunicated. In FIG. 1, the first channel 110 and the second channel 210 are both represented by dotted lines since they are provided internally.

Specifically, the first tube body 100 surrounds to form the first channel 110, and the first channel 110 generally extends along a direction from the proximal end of the surgical instrument to the distal end, and may be used for passage of the inner core of the surgical instrument. It may be understood that in actual use, an extension direction of the first channel 110 is not a fixed direction, but may extend in a path that is straight or curved and is variable in shape, depending on the use state of the first tube body 100. Along the extension direction of the first channel 110, the corresponding two ends of the first tube body 100 are a first end 101 and a second end 102, respectively.

In combination with FIGS. 1 to 4, the first tube body 100 includes a plurality of strands of wires 121, each strand of wire 121 spirally winds from the first end 101 to the second end 102, the plurality of strands of wires 121 spirally wind to form at least one spiral wire harness layer 120, the spiral wire harness layer 120 surrounds to form the first channel 110. That is, the first channel 110 is a channel formed inside the spiral wire harness layer 120.

The second tube body 200 surrounds to form the second channel 210, the second channel 210 is communicated with the first channel 110, and both channels are used for allowing the inner core of the surgical instrument to pass therethrough. Where the second channel 210 also extends in the direction from the proximal end to the distal end. With the change of the use state of the second channel 210, the extension direction of the second channel 210 also presents a straight or curved path with variable shape. Along the extension direction of the second channel 210, the corresponding two ends of the second tube body 200 are a third end 201 and a fourth end 202, respectively. The third end 201 is connected with the second end 102, and the fourth end 202 is used to connect a surgical executing piece located at the most distal end of the surgical instrument. Where a minimum bending radius of the second tube body 200 is smaller than a minimum bending radius of the first tube body 100.

It can be understood that the first tube body 100 of the torque transmission tube 10 includes a plurality of strands of wires 121, which spirally extend and wind to form at least one spiral wire harness layer 120. Such spiral structure of the plurality of strands of wires 121 ensures the torsional strength of the first tube body 100, causing the torque input from the first end 101 may be more effectively transmitted to the second end 102. By providing the second tube body 200 and connecting the third end 201 of the second tube body 200 with the second end 102, the torque of the second end 102 may be effectively transmitted to the third end 201 and then to the fourth end 202 of the second tube body 200 through the third end 201. Therefore, in a first aspect, the torque transmission tube 10 may effectively transmit the torque. Moreover, the minimum bending radius of the second tube body 200 is smaller than the minimum bending radius of the first tube body 100, which means that the second tube body 200 is easier to bend, and the angle may be easily adjusted in a narrow organ cavity. Therefore, in a second aspect, the torque transmission tube 10 has better operation flexibility, which is conducive to improving the operation accuracy of endoscopic instruments and reducing the operation difficulty. The torque transmission tube 10 may be used for the surgical instruments used under the endoscope, and the first channel 110 and the second channel 210 thereof may be used for allowing the inner core of the surgical instruments to pass therethrough. The torque may be transmitted through the torque transmission tube 10, so as to solve the problem of poor torque transmission effect caused by torque transmission through the inner core in the existing surgical instruments.

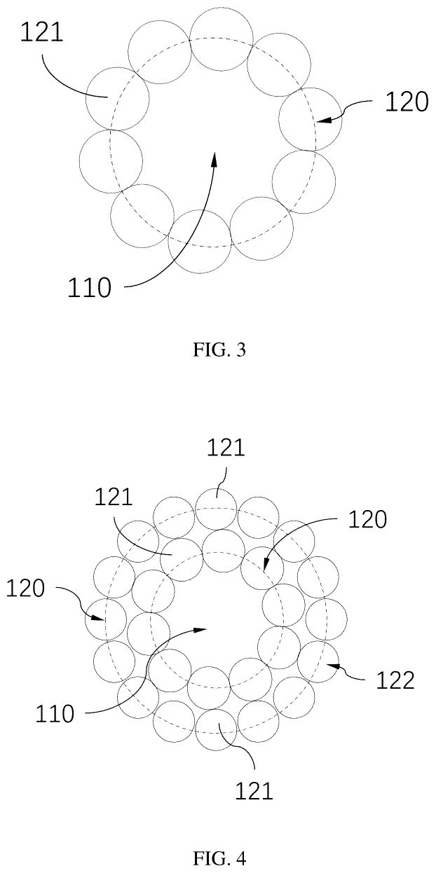

With reference to FIG. 3, in a first embodiment, the first tube body 100 includes one spiral wire harness layer 120, which is formed by a plurality of strands of wires 121, and the plurality of strands of wires 121 are arranged around the first channel 110, and rotate and extend from the first end 101 in a spiral shape and in a same direction to the second end 102. It can be understood that the spiral wire harness layer 120 is a structure formed by winding the plurality of strands of wires 121 in the same direction.

With reference to FIG. 4, in a second embodiment, the first tube body 100 includes a plurality of spiral wire harness layers 120, the plurality of spiral wire harness layers 120 are stacked along a radial direction of the first tube body 100, and each spiral wire harness layer 120 includes at least one strand of wire 121. In two adjacent spiral wire harness layers 120, one spiral wire harness layer 120 surrounds the periphery of the other spiral wire harness layer 120, further enhancing the torsional strength of the first tube body 100, so that the torque may be transmitted better.

It should be noted that when the first tube body 100 includes a plurality of spiral wire harness layers 120, the wires 121 of the spiral wire harness layers 120 each may be the same or different. For example, the spiral wire harness layers 120 each may be formed by spirally winding one strand of wire 121; or they are all formed by spirally winding a plurality of wires 121; or, some layers are formed by spirally winding one strand of wire 121, and other layers are formed by spirally winding a plurality of strands of wires 121. This may be selected according to actual needs.

Further, in a second embodiment, at least two adjacent spiral wire harness layers 120 have wires 121 with different spiral extension directions. For example, when viewed along the extending direction of the first channel 110, in two adjacent spiral wire harness layers 120, the wire 121 of one spiral wire harness layer extends in a clockwise spiral and the wire 121 of the other spiral wire harness layer extends in a counterclockwise spiral. Thus, the torsional strength of the first tube body 100 in both clockwise and counterclockwise directions may be enhanced to ensure a better torque transmission effect.

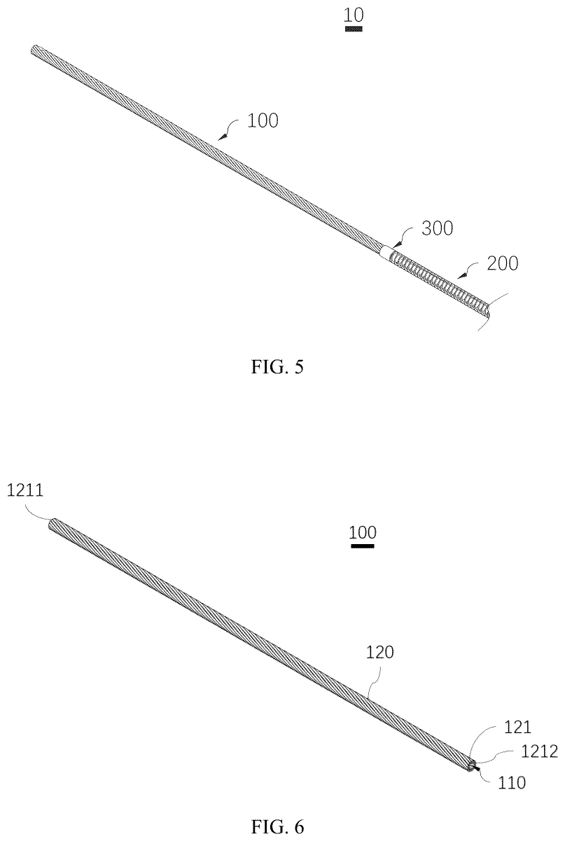

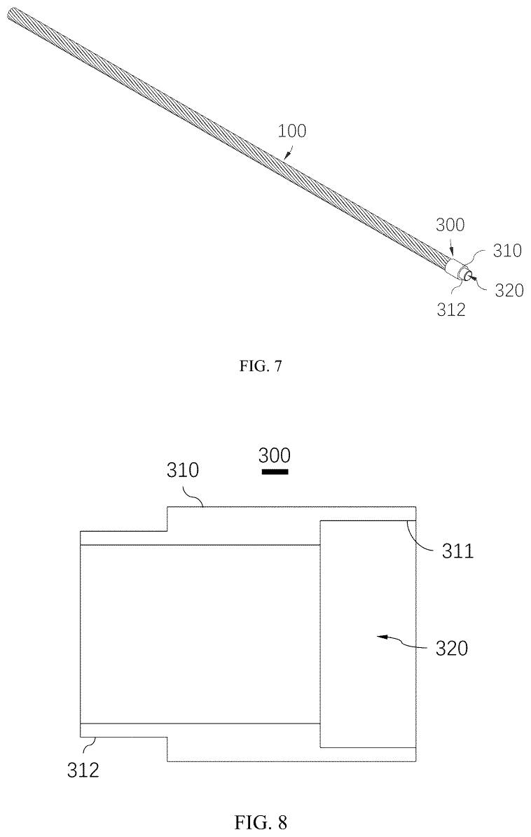

With reference to FIG. 5, FIG. 6 and FIG. 7, this third embodiment has substantially the same structure as the first embodiment, except that, in the third embodiment, the torque transmission tube 10 further includes a connecting piece 300 for connecting the first tube body 100 and the second tube body 200, to facilitate assembly of the torque transmission tube 10. The connecting piece 300 is provided between the first tube body 100 and second tube body 200, and the second end 102 is connected with the third end 201 through the connecting piece 300, so that the first tube body 100 and the second tube body 200 are connected together.

Specifically, the connecting piece 300 includes a body 310 and a third channel 320 passing through the body 310, and a first assembly part 311 and a second assembly part 312 are provided at both ends of the body 310 along an extension direction of the third channel 320. The second end 102 of the first tube body 100 is connected to the first assembly part 311, and the third end 201 of the second tube body 200 is connected to the second assembly part 312, thereby achieving the connection between the first tube body 100 and the second tube body 200. Meanwhile, the third channel 320 is communicated between the first channel 110 and the second channel 210, so as to realize the communication between the first channel 110 and the second channel 210.

Where the first assembly part 311 and the second assembly part 312 may be implemented in a variety of ways. For example, With reference to FIG. 8, the first assembly part 311 may be a groove formed at one end of the body 310, the bottom of the groove is communicated with the third channel 320, and the connection between the first assembly part 311 and the second end 102 is realized by embedding the second end 102 of the first tube body 100 in the groove, and also the binding on the first tube body 100 may be formed by the side wall of the groove, thereby preventing the wire 121 from loosening. For example, the second assembly part 312 may be a plug structure formed at the other end of the body 310, the third channel 320 passes through the plug structure, and by inserting the plug structure from the third end 201 of the second tube body 200 into the second channel 210, the connection between the second assembly part 312 and the second tube body 200 may be achieved, facilitating the mounting and fixing of the two. For another example, the first assembly part 311 and the second assembly part 312 may be assembly grooves formed on two end faces of the body 310, respectively, and the assembly and connection between the first tube body 100 and the second tube body 200 are realized by embedding the first tube body 100 and the second tube body 200 into the assembly grooves.

It should be noted that the first tube body 100 and the second tube body 200 may be directly connected with each other, for example, by welding the second end 102 and the third end 201 into one, and the specific structure will not be repeated here.

In some embodiments, the wire 121 includes a first connector 1211 located at the first end 101 and a second connector 1212 located at the second end 102, the first connectors 1211 of the plurality of wires 121 are connected to each other and the second connectors 1212 of the plurality of wires 121 are connected to each other, which may avoid loosening of the wires 121 during torsion of the first tube body 100, ensure the integrity of the structure, and improve the transmission effect of the torque.

In some embodiments, the extension direction of the first channel 110 is defined as an axial direction of the first tube body 100; under an axial tension of 10 N, an axial tensile displacement of the first tube body 100 does not exceed 5 mm; and under an axial pressure of 60 N, an axial compression displacement of the first tube body 100 does not exceed 40 mm. That is, when the first tube body 100 is tensioned along the axial direction of the first tube body 100 at a tension of 10 N, the deformation of the first tube body 100 under the tension does not exceed 5 mm; and when the first tube body 100 is compressed along the axial direction of the first tube body 100 at a pressure of 60 N, the deformation of the first tube body 100 under the pressure does not exceed 40 mm. Where the axial tension and axial pressure are all applied by a tension machine.

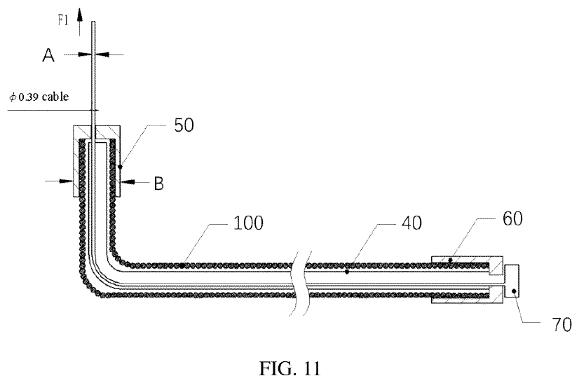

With reference to FIG. 11, this is a compression displacement test, including steps below:
1) fixing one end of a cable with a cable head fixing block 70, and sleeving a plastic tube over the cable;
2) sleeving a lower fixing sleeve 60 and an upper fixing sleeve 50 over both ends of the first tube body 100, respectively;
3) passing one end (free end) of the cable through the lower fixing sleeve 60, and passing it through the upper fixing sleeve 50 along an inner channel of the first tube body 100, leaving a final section of the cable outside (this section is used for subsequent placement on a clamp of the tension machine, i.e., position A);
4) installing the upper fixing sleeve 50 on a lower clamp of the tension machine, and fixing the section of the cable left in step 3) on an upper chuck of the tension machine, while ensuring that the cable is in a tight state;
5) starting the tension machine, with a speed of the tension machine being setting to be 50 mm/min, and when the tensile value reaches 60 N, stopping the tension machine and reading out a movement displacement of the tension machine at this time, i.e., the compression displacement of the first tube body 100.

The tensile displacement is measured by measuring the axial dimension (i.e., length L₁ in FIG. 2) of the first tube body 100 before and after the force is applied. An amount of change of the dimension is the displacement. The test for the tensile displacement includes steps below:
1) setting an initial distance between upper and lower chucks of the tension machine as 500+10 mm;
2) fixing two ends of the first tube body 100 with the length L₁ on the upper chuck and the lower chuck of the tension machine respectively, and ensuring that the first tube body 100 is in a tight state;
3) setting a speed of the tension machine to be 50 mm/min, starting the tension machine, and stopping when the tension machine reaches 10N, and then reading out the movement displacement of the chuck of the tension machine at this time, i.e., the tensile displacement of the first tube body 100.

In the present embodiment, the first tube body 100 of the torque transmission tube 10 meets the above mechanical properties, so that it has more excellent torque transmitting effect, and is more flexible in use of the endoscope.

In some embodiments, in the first tube body 100, the wires 121 have n strands in number, and a diameter of the wires 121 is d mm, meeting: n/d≥1. For example, n/d may be any value of 1, 2, 3, 4, 5, 6, 7, 8, 9, 10, 20, 30, 40, 50, and 100 or a range between any two values. The value of n/d is set in this range so that a tube wall of the first tube body 100 is not too thick, which, on one hand, enables that the tube diameter of the torque transmission tube 10 meets the needs of the surgical instrument without being too large, reducing friction on the inner wall of organ, and on the other hand, enables that the diameter of the first channel 110 may be effectively guaranteed, thereby guaranteeing the assembly space of the inner core and guaranteeing the smooth movement of the inner core. When n/d is less than 1, the number of the wires 121 is relatively small, the diameter of the wires is relatively large, which may occupy the space of the first channel 110, or make the torque transmission tube 10 too thick, and furthermore which is also inconvenient for winding and bonding between the wires 121, resulting in too large clearance between adjacent wires 121. When n/d is greater than 100, the number of the wires is relatively large, the diameter of the wires is relatively thin, and multilayer spiral wire harness layers 120 need to be wound, which is inconvenient for processing and manufacturing. In some embodiments, it is 2≤n≤16, and 0<d≤2.

In some embodiments, the minimum bending radius of the first tube body 100 is r₁, and the minimum bending radius of the second tube body 200 is r₂, meeting: 1<r₁/r₂≤8. For example, r₁/r₂ may be any value among 1.5, 2, 3, 4, 5, 6, 7 and 8 or a range between any two values. When r₁/r₂ is set in this range, the torque of the first tube body 100 may be smoothly transmitted to the distal end through the second tube body 200, and also it is ensured that the angle at the distal end may easily be adjusted in the narrow organ cavity, thereby guaranteeing the operational flexibility. In some embodiments, it is 20 mm≤r₁≤40 mm; 5 mm≤r₂≤20 mm. Where both of the minimum bending radius r₁ of the first tube body 100 and the minimum bending radius 12 of the second tube body 200 are detected by a detection method provided by YY0762-2009 Medical endoscope optical cable for lighting.

With reference to FIG. 2, in some embodiments, a length of the first tube body 100 is L₁, and a length of the second tube body 200 is L₂, meeting: 0<L₂/L₁≤1. For example, L₂/L₁ may be any value among 0.1, 0.2, 0.3, 0.4, 0.5, 0.6, 0.7, 0.8, 0.9 and 1 or a range between any two values. In this range, the larger the value of L₂/L₁ is, the more flexible the distal end of the torque transmission tube 10 is, the easier it is to be bent to adapt to the operation of the doctor in surgery, and accordingly, the improvement of the torque transmission effect is relatively weakened. By controlling the L₂/L₁ within the above range, a better torque transmission effect may be guaranteed, so as to transmit the torque more accurately when in operation of the doctor. The smaller L₂/L₁ is, the stronger the effect enhancement of the torque transmission of the torque transmission tube 10 is, the better the torque transmission effect is, and accordingly, there is a reduction in flexibility of the distal end. In some embodiments, it is 0.3≤L₂/L₁≤0.9.

In some embodiments, a length of the torque transmission tube 10 is L₃, which meets 1550 mm≤L₃≤3000 mm. For example, L₃ may be any value among 1550 mm, 1650 mm, 2350 mm, 2700 mm and 3000 mm or a range between any two values, so as to have a sufficient length to meet the use scenarios of various types of endoscopes.

In some embodiments, the torque transmission tube 10 has a maximum outer diameter of R and a minimum inner diameter of r, meeting: R≤4.2 mm, and r≥0.2 mm. In this way, the torque transmission tube 10 is not too thick, which ensures the adaptability to the organ cavity, ensures the assembly and movement of the inner core, and ensures the application of the torque transmission tube 10 in various types of endoscopes.

In some embodiments, a lead S of a spiral wire formed by a spiral extension of the wire 121 is 3-30 mm, for example, the lead S may be any value among 3 mm, 4 mm, 5 mm, 6 mm, 7 mm, 8 mm, 9 mm, 10 mm, 11 mm, 12 mm, 13 mm, 14 mm, 15 mm, 16 mm, 19 mm, 20 mm, 24 mm, 26 mm, 27 mm, 28 mm, 29 mm, and 30 mm, or a range between any two values. Setting a pitch P within this range enables the finally formed torque transmission tube 10 to have a better torque transmission effect.

With reference again to FIG. 4, in some embodiments, the wire 121 has a cross section 122 perpendicular to the spiral extension direction of the wire; the cross section 122 is circular, or the cross section is polygonal, with four or more sides. By setting the cross section 122 of the wire 121 to the above shapes, adjacent wires 121 after winding may be more closely contacted, ensuring the use effect of the torque transmission tube 10.

Specifically, in some embodiments, materials of the first tube body 100 and the second tube body 200 are both metals. This ensures the overall torsional strength and meanwhile, the service life may also be improved through appropriate metal materials to avoid the rejection from human organs.

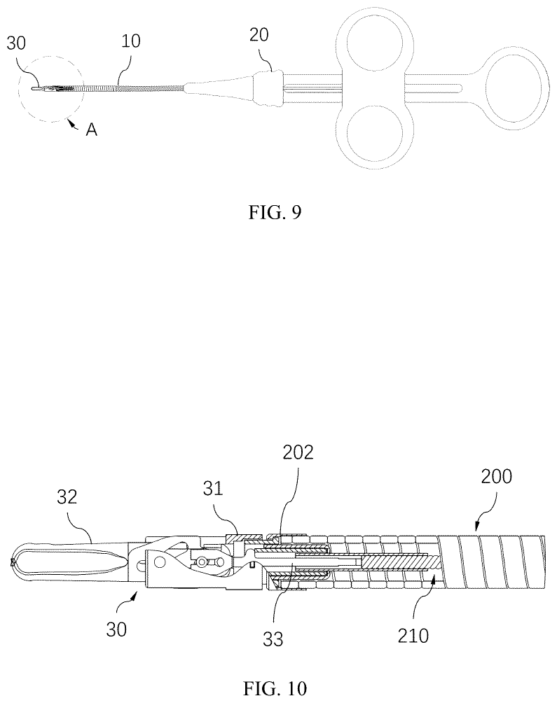

With reference to FIG. 9, an embodiment of the present application also provides a surgical instrument, applied to an endoscope. The surgical instrument includes the torque transmission tube 10 in any one of the above embodiments, an operating handle 20 and an executing piece 30, where the operating handle 20 is connected to the first end 101 for a doctor's operation, and the executing piece 30 is connected to the fourth end 202 for a corresponding action of the operating handle 20 to advance the surgical process; and where the operating handle 20 is used for driving the torque transmission tube 10 to rotate so as to transmit the torque to the executing piece 30. With the torque transmission tube 10 provided in the endoscope, the torque of the operating handle 20 may be transmitted to the executing piece 30 more accurately, and precise control of the executing piece 30 may be realized, thereby reducing the operation difficulty of the doctor, improving the operation efficiency and reducing the operation risk.

Further, with reference to FIG. 10, in some embodiments, the executing piece 30 includes a clamping seat 31, a clamping part 32, and a pulling part 33. The clamping seat 31 is provided at the fourth end 202, the clamping part 32 is movably connected to the clamping seat 31, and the pulling part 33 is movably provided in the first channel 110 and the second channel 210, and is connected between the operating handle 20 and the clamping part 32; where the operating handle 20 is also used for driving the pulling part 33 to move along an extension direction of the pulling part 33, so as to drive the clamping part 32 to move through the pulling part 33, and to separate the clamping part 32 and the clamping seat 31 from the pulling part 33 and the torque transmission tube 10. Specifically, the torque transmission tube 10 may transmit the torque of the operating handle 20 to the clamping seat 31 and the pulling part 33, thereby enabling the entire executing piece 30 to perform a doctor's operation. The clamping part 32 may be a plurality of clips hinged to the clamping seat 31. The pulling part 33 may include a cable connecting with the operating handle 20 and a buckle connecting between the cable and the clamping part 32. The operating handle 20 may drive the pulling part 33 to move along the axial direction of the torque transmission tube 10, thereby driving the clamping part 32 to open or close the clips. Moreover, the operating handle 20 may drive the pulling part 33 to move along the axial direction of the torque transmission tube 10, and may enable the clamping part 32 and the clamping seat 31 to be separated from the pulling part 33 and the torque transmission tube 10 so that they are retained in the patient body to complete the surgical operation.

In the preceding embodiments, the description of each embodiment has its own focus, and the part not detailed in one embodiment may refer to the description of other embodiments.

The torque transmission tube and the surgical instrument provided in the embodiments of the present application are described in detail above, and the principle and the implementations of the present application are explained by applying some specific examples. The description of the preceding embodiments is merely used to help understanding the technical solution and the core idea of the present application. It should be understood by those skilled in the art that it is still possible to modify the technical solutions described in the preceding embodiments, or to equivalently substitute some of the technical features therein. These modifications or substitutions do not make the essence of the corresponding technical solutions out of the scope of the technical solutions of the embodiments of the present application.

What is claimed is:

1. A torque transmission tube, comprising:
a first tube body, having a first channel and corresponding first and second ends in an extending direction of the first channel, wherein the first tube body comprises a plurality of strands of wires, each strand of wire spirally winds from the first end to the second end, the plurality of strands of wires spirally wind to form at least one spiral wire harness layer, the at least one spiral wire harness layer surrounds to form the first channel;
a second tube body, having a second channel and corresponding third and fourth ends in an extension direction of the second channel, wherein the third end is connected with the second end, and the second channel is communicated with the first channel;
wherein a minimum bending radius of the second tube body is smaller than a minimum bending radius of the first tube body.

2. The torque transmission tube according to claim 1, wherein the first tube body comprises one spiral wire harness layer, the plurality of strands of wires are arranged around the first channel and extend in a same direction to form the one spiral wire harness layer.

3. The torque transmission tube according to claim 1, wherein the first tube body comprises a plurality of spiral wire harness layers, the plurality spiral wire harness layers are stacked along a radial direction of the first tube body, and each spiral wire harness layer comprises at least one strand of wire.

4. The torque transmission tube according to claim 3, wherein the wires of at least two adjacent spiral wire harness layers have different spiral extension directions.

5. The torque transmission tube according to claim 1, further comprising:
a connecting piece, provided between the first tube body and the second tube body, and the second end is connected with the third end through the connecting piece.

6. The torque transmission tube according to claim 1, wherein the wire comprises a first connector located at the first end and a second connector located at the second end, the first connectors of the plurality of strands of wires are connected with each other, and the second connectors of the plurality of strands of wires are connected with each other.

7. The torque transmission tube according to claim 1, wherein the extending direction of the first channel is an axial direction of the first tube body;
under an axial tensile force of 10 N, an axial tensile displacement of the first tube body does not exceed 5 mm; and
under an axial pressure of 60 N, an axial compression displacement of the first tube body does not exceed 40 mm.

8. The torque transmission tube according to claim 1, wherein there are n strands of wires, a diameter of the wires is d mm, meeting: n/d≥1.

9. The torque transmission tube according to claim 8, wherein the torque transmission tube further meets:
$$2 \leq n \leq 16 ; and 0 < d \leq 2 .$$

10. The torque transmission tube according to claim 1, wherein the minimum bending radius of the first tube body is r₁, and the minimum bending radius of the second tube body is r₂, meeting: 1<r₁/r₂≤8.

11. The torque transmission tube according to claim 10, wherein the torque transmission tube further meets:
$$20 mm \leq {r}_{1} \leq 40 mm ; and 5 mm \leq {r}_{2} \leq 20 mm .$$

12. The torque transmission tube according to claim 1, wherein a length of the first tube body is L₁, and a length of the second tube body is L₂, meeting: 0<L₂/L₁≤1.

13. The torque transmission tube according to claim 1, wherein a length of the torque transmission tube is L₃, meeting 1550 mm≤L₃≤3000 mm; and/or,
a maximum outer diameter of the torque transmission tube is R and a minimum inner diameter thereof is r, meeting: R≤4.2 mm, and r≥0.2 mm.

14. The torque transmission tube according to claim 1, wherein a lead S of a spiral wire formed by a spiral extension of the wire is 3-30 mm.

15. The torque transmission tube according to claim 1, wherein the wire has a cross section perpendicular to a spiral extension direction of the wire;
the cross section is circular, or,
the cross section is polygonal, the polygon having four or more sides.

16. The torque transmission tube according to claim 1, wherein materials of the first tube body and the second tube body are both metal.

17. A surgical instrument, applied to an endoscope, the surgical instrument comprising:
the torque transmission tube according to claim 1;
an operating handle, provided at the first end; and
an executing piece, provided at the fourth end;
wherein the operating handle is used for driving the torque transmission tube to rotate so as to transmit a torque to the executing piece.

18. The surgical instrument according to claim 17, wherein the first tube body comprises one spiral wire harness layer, the plurality of strands of wires are arranged around the first channel and extend in a same direction to form the one spiral wire harness layer.

19. The surgical instrument according to claim 17, wherein the first tube body comprises a plurality of spiral wire harness layers, the plurality spiral wire harness layers are stacked along a radial direction of the first tube body, and each spiral wire harness layer comprises at least one strand of wire.

20. The surgical instrument according to claim 17, wherein the executing piece comprises:
a clamping seat, provided at the fourth end;
a clamping part, connected with the clamping seat; and
a pulling part, movably provided in the first channel and the second channel, and connected between the operating handle and the clamping part;
wherein, the operating handle is further used for driving the pulling part to move along an extending direction of the pulling part, so as to drive the clamping part to move, and to separate the clamping seat and the clamping part from the torque transmission tube and the pulling part.
